(19) United States
(12) Patent Application Publication
(10) Pub. No.: US 2026/0060775 A1
(43) Pub. Date: Mar. 5, 2026

(54) OMNI-VIEW UNIQUE TRACKING MARKER

(71) Applicant: See All AI Inc., Nashua, NH (US)

(72) Inventors: Eugene Alma Gregerson, North Salt Lake, UT (US); Todd Joseph Furlong, Goffstown, NH (US); Michael Craig Barrus, Redondo Beach, CA (US); Ryan Gregory Woods, Broomfield, CO (US); Eric David Major, Purcellville, VA (US); Stephen William Lang, Hollis, NH (US); Thomas Michael Thornton, Harvard, MA (US)

(73) Assignee: See All AI Inc., Nashua, NH (US)

(21) Appl. No.: 19/204,187

(22) Filed: May 9, 2025

Related U.S. Application Data
(63) Continuation-in-part of application No. 18/974,434, filed on Dec. 9, 2024, now abandoned.
(60) Provisional application No. 63/689,154, filed on Aug. 30, 2024.

(51) Int. Cl. A61B 90/00

(52) U.S. Cl. CPC A61B 90/39; A61B 2090/3937; A61B 2090/3983; A61B 2090/3991

(57) ABSTRACT

A system for temporarily securing a reference marker on the skin of a patient or above the skin of a patient at user selectable heights during a surgical or diagnostic procedure comprises a reference marker, a surgical anchor and an adjustment key. The marker includes a marker body having an exterior profile within a three-dimensional space as measured along three axes, wherein the exterior profile of the marker body is not symmetric relative to any two of the three axes. The anchor includes an anchor shaft and a plurality of attachment features disposed along the anchor shaft between first and second ends thereof, wherein each respective attachment feature provides a point of attachment for the reference marker to the anchor shaft at a different respective length relative to one of the first or second ends. The adjustment key enables the reference marker to be secured at the desired height of the anchor shaft. Also disclosed is a reference marker having an adhesive on a surface thereof for securing directly to the skin of a patient.

### CROSS REFERENCE TO RELATED APPLICATIONS

This application claims the benefit of priority to the following applications, filed by the same Applicant, See All AI Inc., the entire contents of all of which are incorporated herein by this reference for all purposes:

U.S. Patent Application No. 18/974434, entitled “OMNI-VIEW UNIQUE TRACKING MARKER” filed Dec. 9, 2024, and

U.S. Provisional Application No. 63/689154, entitled “INDEPENDENTLY TRACKABLE ANATOMY REFERENCE MARKERS” filed Aug. 30, 2024.

### FIELD OF THE INVENTION

The field of the invention relates to position detection and imaging technology, and, more particularly, to a reference marker designed for accurate visual recognition from any viewing angle within a surgical environment.

### BACKGROUND OF THE INVENTION

Attempts have been made to utilize both radiographic images and visually acquired positional data to assist with surgical navigation, such as the system disclosed in US Patent Application Publication US20210169504A1, however, such system is not capable of creating a three dimensional volume of CT quality images useful for real time surgical navigation purposes. The difficulty in attempting to utilize visually acquired position information and radiographic images is the calibration of the camera's coordinate system with that of the X-ray imaging system. This problem is further compounded when trying to align the position of a surgical instrument as defined within the coordinate system of the patient or camera within the coordinate system of a three dimensional volume of radiographic images, such as CT images.

Accordingly, a need exists for a system and method which is capable of accurately creating a 3D volume of the patient anatomy in an efficient, near real-time manner.

A further need exists for a surgical reference maker that is characterized by its distinct geometry, and texture, ensuring that the reference marker appears unique from every viewing angle.

An even further need exists for a surgical reference maker that has a geometrically distinct shape designed for detection by visible and non-visible light cameras and an exterior surface texture with high-contrast properties relative to human tissue under visible light and non-visible light cameras.

Yet a further need exists for a surgical reference maker that is easy attachable to the anatomy of a subject.

### SUMMARY OF THE INVENTION

A system for temporarily securing a reference marker on the skin of a patient or above the skin of a patient at user selectable heights during a surgical or diagnostic procedure comprises a reference marker, surgical anchor adjustment key. The marker includes a marker body having an exterior profile within a three-dimensional space as measured along three axes, wherein the exterior profile of the marker body is not symmetric relative to any two of the three axes. The anchor includes an anchor shaft and a plurality of attachment features disposed along the anchor shaft between first and second ends thereof, wherein each respective attachment feature provides a point of attachment for the reference marker to the anchor shaft at a different respective length relative to one of the first or second ends. The adjustment key enables the reference marker to be secured at the desired height of the anchor shaft.

Disclosed is a unique reference marker characterized by its distinct geometry, and texture, ensuring that the reference marker appears unique from every viewing angle. The reference marker design enables precise and consistent tracking by visible light cameras in various applications, including motion capture, augmented reality, and automated monitoring systems. The disclosed reference marker has a geometrically distinct shape designed for detection by visible and non-visible light cameras and an exterior surface texture with high-contrast properties relative to human tissue under visible light and non-visible light cameras. The reference marker includes a mechanism for securing placement thereof to a patient anatomy at various user selectable heights from the anatomy. Specifically, a securing mechanism is provided for attaching the marker body at or above skin of a patient. In practice, one or more reference markers may be placed or secured to the patient's body. Visible light cameras are used to capture the imaging data, e.g. the shape and position of the reference marker, and the captured imaging data analyzed to locate the pose of the patient, as indicated by the reference marker, within the space of the patient. In embodiments, the anchor shaft is rigidly attachable directly or indirectly to an anatomical landmark such a vertebrae while the reference marker is movably attachable to the anchor. In this manner the user may selectively adjust the height of the reference marker above the skin while not moving the point of attachment to the patient anatomy.

According to one aspect of the disclosure, a reference marker for imaging procedures, comprises: a marker body comprising a plurality of surfaces defining an exterior profile within a three-dimensional space as measured along three axes, wherein the exterior profile of the marker body is not symmetric relative to any two of the three axes. In some implementations, the reference marker further comprises a securing mechanism for securing the marker body proximate to a surface. In some implementations, the securing mechanism comprises an aperture extending through the marker body. In some implementations, the aperture is adapted to receive and retain a portion of an anchor herein. In some implementations, the securing mechanism comprises a locking mechanism integrally formed with the marker body. In some implementations, the locking mechanism frictionally engages a complimentary feature of an anchor. In some implementations, the locking mechanism comprises an adhesive disposed on the marker body. In some implementations, the marker body comprises material having any of color, luminance, or reflectance properties to increase visual contrast of the marker body relative to biologic tissue. In some implementations, the reference marker is combined with an anchor apparatus.

According to another aspect of the disclosure, a reference marker securable at user selectable heights from a patient's skin, the reference marker comprises: a marker body defining an exterior profile within a three dimensional space as measured along three perpendicular axes, wherein the exterior profile of the marker body is not symmetric relative to two of the three perpendicular axes; and a securing mechanism for attaching the marker body at or above skin of a patient.

According to yet another aspect of the disclosure, an anchor apparatus for use with a reference marker in a surgical procedure, the anchor comprises: an anchor shaft having a first end and a second end; a plurality of attachment features disposed along the anchor shaft between the first and second ends thereof; wherein each respective attachment feature provides a point of attachment to the anchor shaft at a different respective user selectable height above skin of a patient.

According to another aspect of the disclosure, A method for identifying a point of interest in medical imaging procedures, comprises: A) securing a reference marker at or above skin of a patient, the reference marker having an exterior profile which is not symmetric relative to any two dimension in three dimensional space; and B) detecting a position of the reference marker.

According to still another aspect of the disclosure, a system for identifying a point of interest in a medical procedure, the system comprises: reference marker having a marker body which is not symmetric relative to any two dimension in three dimensional space and a securing mechanism for attaching the marker body at or above skin of a patient; and an anchor having a shaft and a plurality of attachment features disposed along the anchor shaft, wherein each respective attachment feature provides a possible point of attachment of the reference marker to the anchor shaft at a different user selectable height from the skin of the patient.

According to another aspect of the disclosure, an anchor apparatus for use with a reference marker comprises: an anchor shaft having first and second ends; and a plurality of attachment features disposed along the anchor shaft between the first and second ends thereof; wherein each respective attachment feature provides a point of attachment to the anchor shaft at a different respective length relative to one of the first or second ends. In some implementations, the plurality of attachment features comprise a plurality of apertures extending through the anchor shaft. In some implementations, the plurality of apertures are substantially rectangular in shape. In some implementations, the plurality of attachment features have equal spacing therebetween. In some implementations, the plurality of attachment features comprise a plurality of detents in the anchor shaft. In some implementations, the plurality of detents have equal spacing therebetween. In some implementations, the anchor shaft has a substantially circular cross-sectional shape. In some implementations, the anchor apparatus is combined with an reference marker.

According to still another aspect of the disclosure, a reference marker for medical imaging procedures comprises: a marker body having a geometrically unique exterior profile detectable by visible and non-visible light cameras and made from material having high-contrast properties relative to biologic tissue under visible light; and a mechanism for securing the reference marker to a patient anatomy, wherein the reference marker is configured to be distinctly identifiable by visible and non-visible light cameras.

According to even another aspect of the disclosure, a method for enhancing accuracy in medical imaging procedures comprises: A) placing a reference marker on a surface of interest, the reference marker comprising a marker body having a plurality of surfaces defining a unique exterior profile within a three dimensional space as measured along three perpendicular axes, wherein the exterior profile of the marker body is not symmetric relative to any two of three axes; and B) capturing image data of the reference marker using visible or non-visible light cameras. In some implementations, A) comprises: A1) placing a reference marker in a predetermined position of a patient's anatomy relative to an area of interest. In some implementations, A) comprises: A1) securing the reference marker to an anchor, and A2) securing the anchor to a patient's anatomy.

According to still another aspect of the disclosure, a reference marker securable to a patient's skin or at user selectable heights from a patient's skin, the reference marker comprises: a marker body comprises material having any of color, luminance, or reflectance properties to increase visual contrast of the marker body relative to biologic tissue, a first hollow shaft extending through the marker body and configured for slidably receiving a surgical anchor shaft therein; and a second hollow shaft extending through the marker body and in communication with the first hollow shaft and configured for movably receiving a securing device therein.

According to still another aspect of the disclosure, a system for identifying a point of interest in a medical procedure, the system comprises: reference marker having a marker body which is not symmetric relative to any two dimension in three dimensional space; an anchor rigidly couplable to an anatomical landmark, the anchor comprising a shaft and a plurality of attachment features disposed along the anchor shaft, wherein each respective attachment feature provides a possible point of attachment of the reference marker to the anchor shaft at a different user selectable heights above skin of the patient; a securing mechanism for attaching the marker body at a different user selectable heights above skin of the patient; and a mechanism for releasably engaging and disengaging the securing mechanism to the anchor shaft.

According to still another aspect of the disclosure, a system for securing a reference marker to a patient's skin or at user selectable heights from a patient's skin, the system comprises: an anchor rigidly couplable to an anatomical landmark, the anchor comprising a shaft extending along an axis; and a reference marker comprising a marker body having an aperture extending therethrough for slidably receiving the anchor shaft, the marker body comprising a material having any of color, luminance, or reflectance properties to increase visual contrast of the marker body relative to biologic tissue; wherein the marker body is slidably movable and securable along the anchor shaft axis while the anchor is rigidly attached to the anatomical landmark. In embodiments, the system further comprises a locking mechanism for securing the marker body to the anchor shaft. In embodiments, the locking mechanism comprises an aperture extending through the marker body. In embodiments, the locking mechanism further comprises a screw movable mounted within the marker body. In embodiments, the locking mechanism frictionally engages the anchor shaft. In embodiments, the system of claims 2 further comprising a key removably engagable with the locking mechanism. In embodiments, the key and the locking mechanism have complimentary mating engagement features.

According to yet another aspect of the disclosure, a reference marker securable to a patient's skin or at user selectable heights from a patient's skin, the reference marker comprises: a marker body comprises material having any of color, luminance, or reflectance properties to increase visual contrast of the marker body relative to biologic tissue, a first hollow shaft extending through the marker body and configured for slidably receiving a surgical anchor shaft therein; and a second hollow shaft extending through the marker body and in communication with the first hollow shaft and configured for movably receiving a securing device therein. In embodiments, the reference marker is in combination with surgical anchor. In embodiments, the surgical anchor is rigidly couplable to an anatomical landmark, the anchor comprising a shaft and a plurality of attachment features disposed along the anchor shaft, wherein each respective attachment feature provides a possible point of attachment of the reference marker to the anchor shaft at a different user selectable heights above skin of the patient. In embodiments, the securing device and the second hollow shaft have complimentary mating features on respective exterior surfaces thereof. In embodiments, the second shaft has a threaded exterior and the shaft and the securing device comprises a threaded screw movable disposed within the second shaft. In embodiments, the reference marker is in combination with a key receivable within an end of the screw for advancing or retracting the screw within the second shaft. In embodiments, the anchor shaft and first hollow shaft have a substantially circular cross-sectional shape.

According to another aspect of the disclosure, a method for identifying a point of interest in medical imaging procedures, comprises: A) rigidly coupling an anchor to an anatomical landmark, the anchor comprising a shaft extending along an axis; B) securing a reference marker to the anchor shaft at or above skin of a patient, a reference marker comprising a marker body having an aperture extending therethrough for slidably receiving the anchor shaft, the marker body comprising a material having any of color, luminance, or reflectance properties to increase visual contrast of the marker body relative to biologic tissue; and C) detecting a position of the reference marker. In embodiments, A) comprises: A1) securing the anchor in a position of a patient's anatomy proximate a surgical entry point. In embodiments, the marker body further comprises a securing mechanism movably mounted therein and A) further comprises: B1) securing the reference marker to the anchor shaft with a key mechanism by advancing the securing mechanism through the marker body until contacting the anchor shaft.

A system for identifying a point of interest in a medical procedure, the system comprises: an anchor rigidly couplable to an anatomical landmark, the anchor comprising a shaft extending along an axis and further a plurality of attachment features disposed along the anchor shaft, wherein each respective attachment feature provides a possible point of attachment of the reference marker to the anchor shaft at a different user selectable heights above skin of the patient; reference marker comprising a marker body having an aperture extending therethrough for slidably receiving the anchor shaft; securing mechanism movably disposed within the marker body for attaching the marker body at a different positions along the axis of the anchor shaft. In embodiments, the securing mechanism frictionally engages the anchor shaft. In embodiments, the securing mechanism comprises a screw disposed within a threaded shaft in the marker body, the threaded shaft configured to movably receive the screw therein. In embodiments, the system is in combination with a key mechanism for releasably engaging and disengaging the screw to or from the anchor shaft. In embodiments, the key mechanism and screw have complimentary mating features on respective exterior surfaces thereof.

### DESCRIPTION OF THE DRAWINGS

The various features and advantages of the disclosure may be more readily understood with reference to the following detailed description taken in conjunction with the accompanying drawings, wherein like references designate like structural elements, and in which:

FIG. 1 illustrates conceptually a top perspective view a reference marker in accordance with the disclosure;

FIG. 2 is a top view of the reference marker of FIG. 1 in accordance with the disclosure;

FIG. 3 is a bottom view of the reference marker of FIG. 1 in accordance with the disclosure;

FIG. 4 is a front view of the reference marker of FIG. 1 in accordance with the disclosure;

FIG. 5 is a rear view of the reference marker of FIG. 1 in accordance with the disclosure;

FIG. 6 is a side plan view of the reference marker of FIG. 1 in accordance with the disclosure;

FIG. 7 is a wire view of the exemplary plan view of the reference marker in accordance with the disclosure as attached to a portion anatomy by an anchor;

FIG. 8 illustrates conceptually a top perspective view a reference marker in accordance with the disclosure;

FIG. 9 illustrates conceptually a top view of the reference marker in accordance with the disclosure as attached to a portion anatomy by an anchor;

FIG. 10 illustrates conceptually a top perspective view of a reference marker with an adhesive securing mechanism in accordance with the disclosure;

FIG. 11 illustrates a bottom perspective view of a reference marker with an adhesive securing mechanism in accordance with the disclosure;

FIG. 12 illustrates conceptually a top view a reference marker in accordance with the disclosure as placed on a patient anatomy;

FIG. 13 is a side view of anchor useable with the reference marker of FIG. 1 in accordance with the disclosure;

FIG. 14 is a front view of the reference marker of FIG. 1 secured to the anchor of FIG. 13 in accordance with the disclosure;

FIG. 15 is a side view of anchor in accordance with the disclosure;

FIG. 16 is a side view of anchor in accordance with the disclosure;

FIG. 17A is a top perspective view of the reference marker of FIG. 16 secured to the anchor of FIG. 16 in accordance with the disclosure;

FIG. 17B is a side perspective view of the reference marker of FIG. 16 secured to the anchor of FIG. 16 in accordance with the disclosure;

FIG. 18 is a conceptual illustration of a surgical navigation system suitable for use with the reference markers and anchors described in accordance with the disclosure;

FIG. 19 is a perspective view of a reference marker secured to an anchor in accordance with the disclosure;

FIG. 20 is a perspective, exploded view of the reference marker and anchor of FIG. 19 in accordance with the disclosure;

FIG. 21 is a full length perspective view of the reference marker and anchor of FIG. 19 secured together in accordance with the disclosure;

FIG. 22 is a perspective view of a system including a marker, anchor and key in accordance with the disclosure; and

FIG. 23 is a cross sectional view of the reference marker, anchor, and key conjoined in accordance with the disclosure.

### DETAILED DESCRIPTION

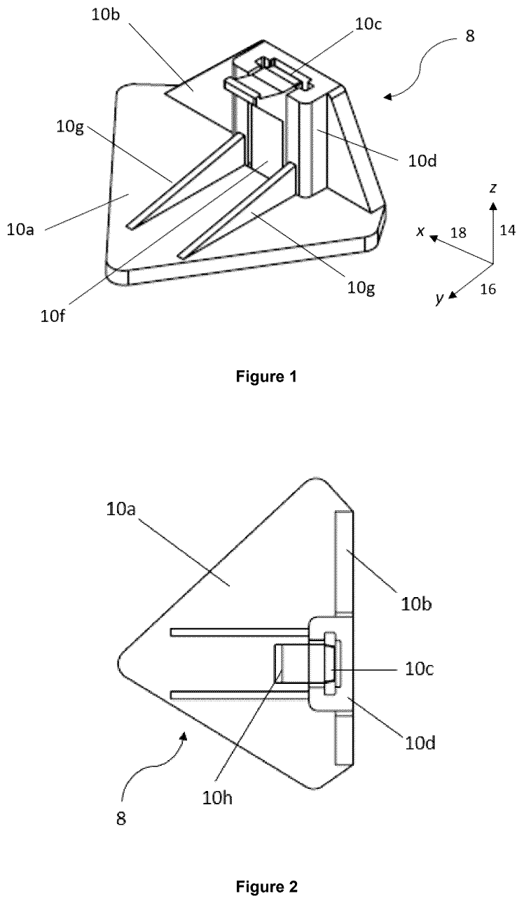

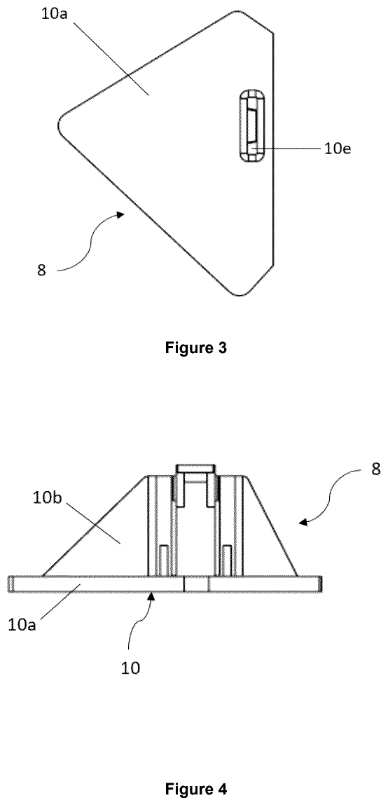

FIG. 1 illustrates an exemplary embodiment of the reference marker 8 in accordance with the disclosure. The disclosed marker 8 comprises a geometrically complex, marker body 10, and a securing mechanism for removably attaching marker 8 to a patient's anatomy or other object of interest. Marker body 10 is designed to present a unique profile from possible viewing angles. In some implementations, the marker body 10 can be any shape comprising a plurality of surfaces defining an exterior profile within a three-dimensional co-ordinate space as defined by axis 14, 16, and 18, where the exterior profile of the marker body 10 is not symmetric relative to any two of the three axis 14, 16, and 18, as described herein. In some implementations, the exterior profile of the marker body 10 is not symmetric relative to any one of the three axis 14, 16, and 18, as described herein. Lack of symmetry of the surfaces comprising marker body 10 contributes to the overall detectability for the reference marker 8 to be visually distinguished by a camera. A marker body 10 which is asymmetric in all three dimensions, as referenced by axis 14, 16, and 18, highly trackable and detectable within a camera visual coordinate system. A marker body 10 which is asymmetric in two of three dimensions, as referenced by axis 14, 16, and 18, is also trackable and detectable within a camera visual coordinate system. A marker body 10 which is asymmetric in only one of three dimensions, as referenced by axis 14, 16, and 18, is less trackable and detectable within a camera visual coordinate system. Further, although the embodiment illustrated in FIGS. 1-6 show a reference marker 8 with a marker body 10 comprising primarily planar surfaces, the marker body 10 may comprises one or more non-planar, e.g. curved, surfaces as well. However, the number of non-planar surfaces as well as an increased angle of curvature may negatively affect how trackable and detectable within a camera visual coordinate system.

In some implementations, the surfaces of marker body 10 has multiple facets or surfaces which may be manufactured or textured to affect the reflectivity of the surface, as illustrated in FIGS. 1-6. When viewed by a color or visible light camera, the unique geometry, lack of symmetry, and/or surface textures of marker 8 enable the marker to be easily distinguished from its surroundings, regardless of a camera angle(s), to enable precise tracking of the position and orientation of marker 8 in a three-dimensional space.

In some implementations, the marker 8 may be made of a low-cost, substantially rigid material such as plastic or other synthetic resins which are capable of maintaining the marker shape and being manufactured in a sterile environment. The textured surfaces of marker body 10 may be formed during manufacturing, i.e. through injection molding, or as a separate process thereafter through any of chemical, mechanical, or other treatment processes. In some implementations, in addition to the unique shape of marker 8, increased visibility of marker 8 may be achieved through a combination of colors, luminance, or reflective materials.

In some implementations, the marker body 8 has an asymmetric primary surface 10a and an asymmetric support wall 10b which extends outwardly therefrom at a right angle to primary surface 10a. Wall 10b flanks a U-shaped support projection 10d partially surrounding an aperture 10e extending though primary surface 10a. Aperture 10e is adapted to receive an anchor 30 therethrough for positioning marker 8 at or above a surface of interest. e.g. a surgical entry point on a patient, as explained in greater detail herein.

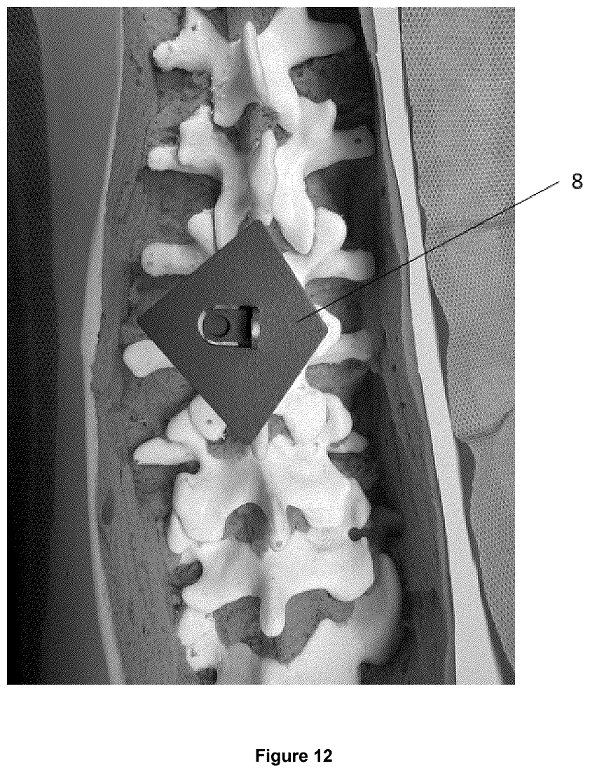

A mechanism 10f for securing marker 8 to an anchor 30, as explained herein, comprises a projection flexibly hinged and integrally formed at one end thereof to the marker body 10. The free end of the projection includes a thumb tab 10h opposite a pivoted catch or pawl 10c designed to frictionally engage any of a notch, detent, indentation, aperture or other feature on anchor shaft 30a so as to allow movement of the marker 8 relative to the anchor 30 only when locking mechanism 10f is pivoted or counter biased away from the feature on anchor shaft 30e with which it is engaged. The design of the securing mechanism 10f may be determined in part by the design of anchor 30. The aperture 10e should be sized and shaped to pass but retain therein the exterior profile of anchor shaft 30e as described herein. A pair of secondary support walls 10g extending outwardly and at a right angle to primary planar surface 10a provide further support to U-shaped support projection 10d. FIG. 12 illustrates conceptually a top view a spatial orientation reference marker in accordance with the disclosure as placed on a patient anatomy.

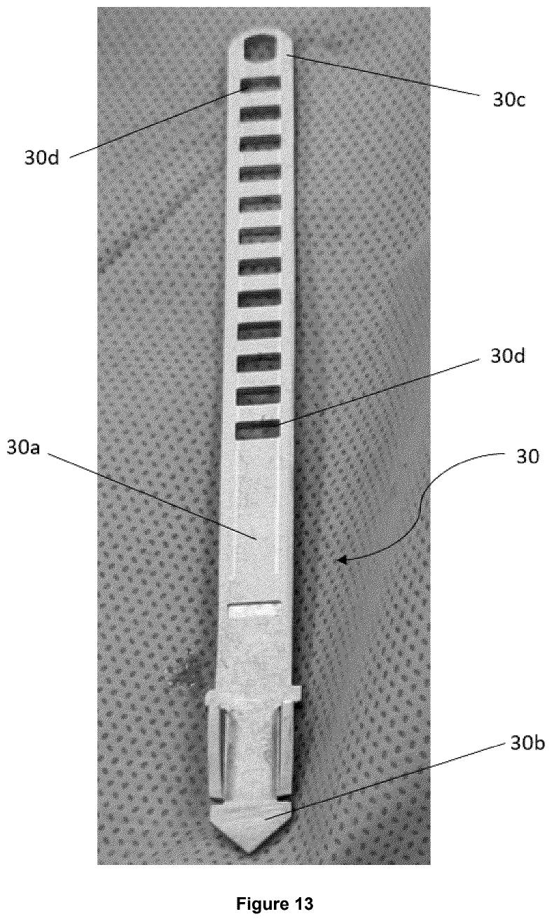

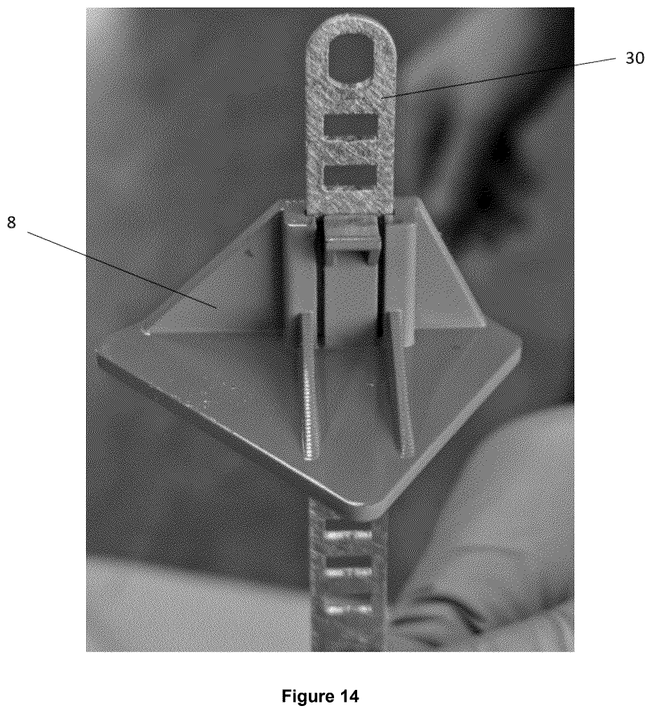

FIG. 13 illustrates an exemplary anchor 30 to which marker 8 may be secured. In some implementations, anchor 30 may be implemented with a substantially elongate shaft 30a extending between ends 30b and 30c. End 30b has a tapered shape for penetrating surfaces and may or may not have additional reinforcements features thereat for strengthening end 30b. In some implementations, end 30c has an aperture which forms a loop for easy grasping and removal of the anchor 30 and/or marker 8 at the end of a procedure. A plurality of features 30d, to which securing mechanism 10e may interact, are disposed along shaft 30a intermediate ends 30b and 30c. In some implementations, elongate shaft 30a is substantially flat, however, the elongate shaft 30a may have any cross-sectional shape including, but not limited to, circular or rectangular, or other geometric or irregular shapes. In some implementations, features 30d may be implemented with substantially rectangular apertures extending through shaft 30a and with substantially equal spacing therebetween. In some implementations, other shaped aperture and/or features may extend all or partially through shaft 30a with locations and spacing therebetween that are the same or different than as illustrated herein. In some implementations, anchor 30 may be made from a substantially rigid material having adequate columnar strength, such as surgical stainless, metals or rigid plastics, to penetrate at least soft tissue. FIG. 14 illustrates a marker 8 attached to anchor 30.

In practice, one or more reference markers are placed or secured to the patient's body. Specifically, placing a reference marker comprises securing the reference marker to an anchor and securing the anchor in a patient anatomy relative to a predetermined area of interest for the medical imaging procedure, and capturing medical imaging data of the reference marker using visible light cameras. Visible light cameras are used to capture the imaging data, e.g. the shape and position of the reference marker, and the captured imaging data analyzed to locate the pose of the patient, as indicated by the reference marker, within the space of the camera coordinate space. In some implementations, the anchor may be secured to the patient anatomy and the reference marker then secured to the anchor. In some implementations, the reference marker may also be secured to the patient anatomy by removing the tab 10g protecting the adhesive and then securing the surface 10a to the patient anatomy.

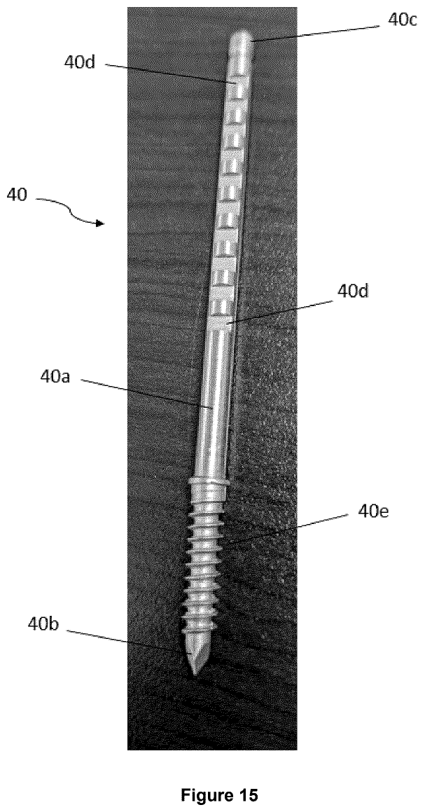

FIG. 15 illustrates an exemplary anchor 40 to which marker 8 may be secured. In some implementations, anchor 40 may be implemented with a substantially elongate shaft 40a extending between ends 40b and 40c. In some implementations, elongate shaft 40a has a substantially circular cross-sectional shape. End 40b has a distal tapered tip for penetrating harder surfaces, e.g. bone, and a more proximal area 40e having a screw threaded exterior. In some implementations, elongate shaft 40a is substantially solid with a plurality of features 40d to which securing mechanism 10e of marker 8 may interact, disposed along shaft 40a intermediate ends 40b and 40c. In some implementations, features 40d may be implemented with substantially rectangular notches or detents extending partially into shaft 40a and with substantially equal spacing therebetween. In some implementations, anchor 40 may be made from a substantially rigid material having adequate columnar strength, such as surgical stainless, metals or rigid plastics, to penetrate at least soft tissue and/or bone as necessary. In some implementations, other end configurations may be employed to so that the anchor may interact with other surgical hardware.

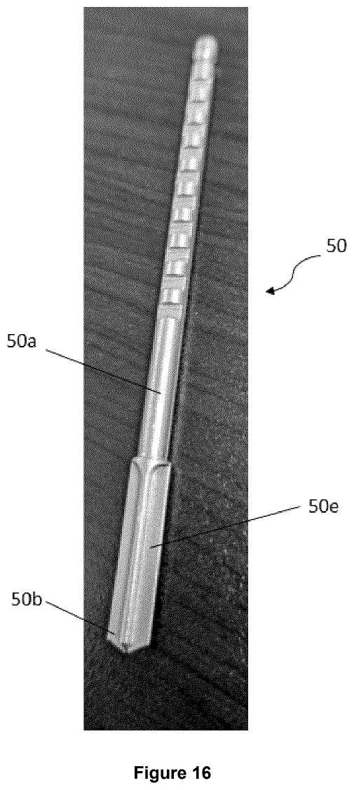

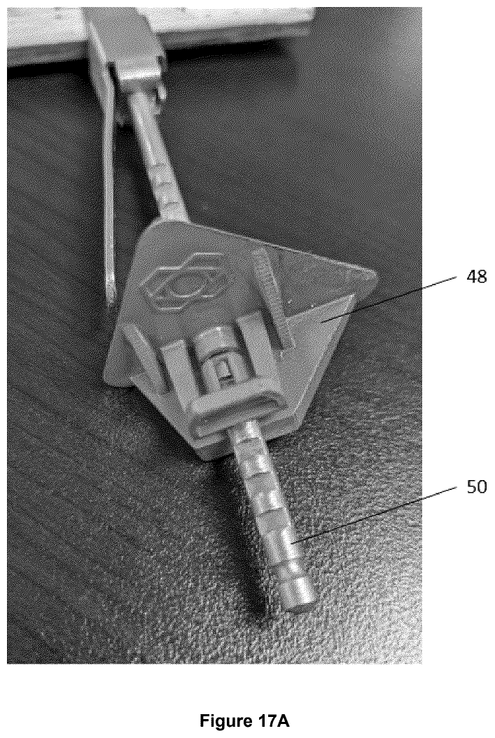

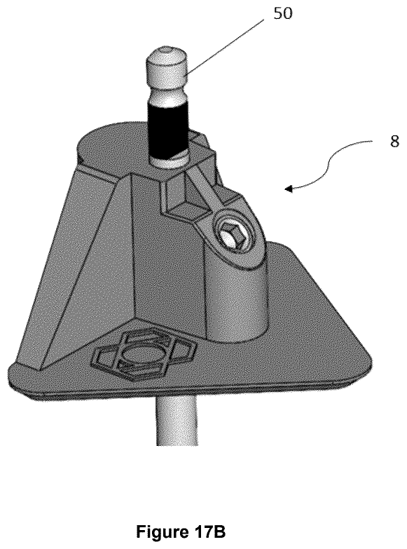

FIG. 16 is a side view of an anchor 50 in accordance with the disclosure. In some implementations, anchor 50 may be implemented substantially the same as anchor 40 except that end 50b and area 50e of elongate shaft 50a are implemented with a coupling features that enable end 50b to interact with other surgical hardware. FIG. 17 is a top view of the reference a marker 48 secured to anchor 50 of FIG. 16 in accordance with the disclosure. Marker 48 may be implemented substantially the same as anchor 8 except that securing mechanism 10f, 10c and aperture 10e of marker 8 are modified in marker 50 to accommodate the circular cross-sectional shape of either shaft 40a or 50a.

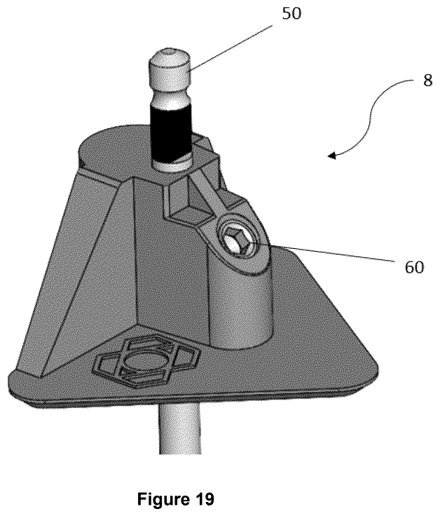

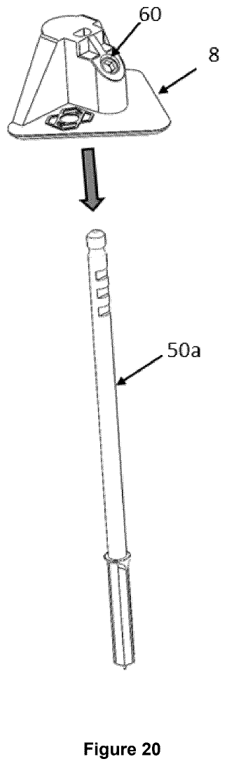

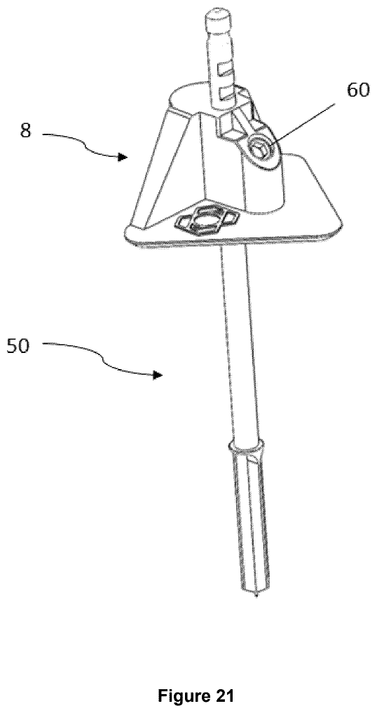

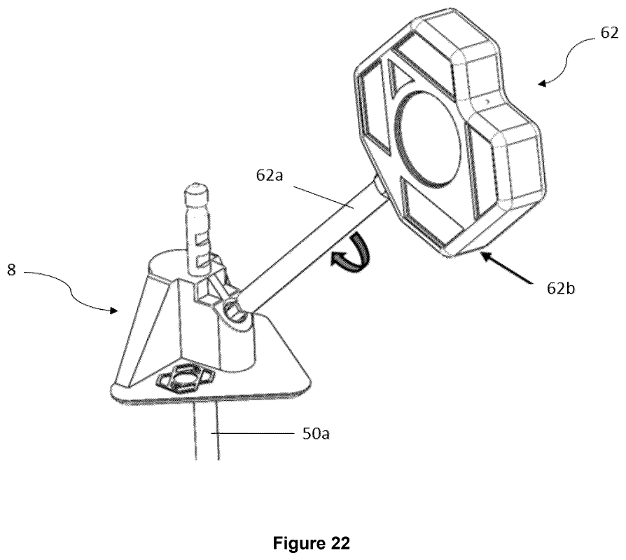

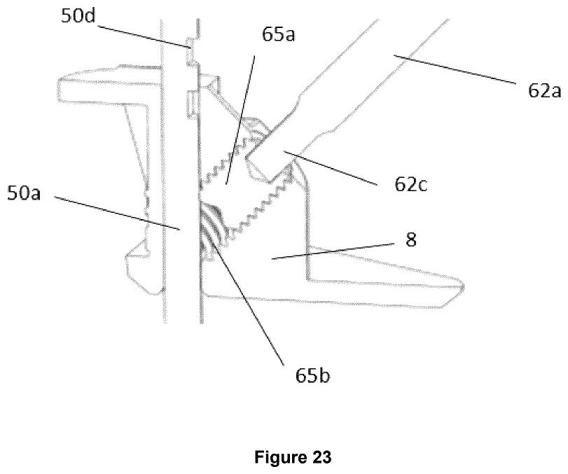

A system 55 for securing a reference marker 58 to a patient's skin or at user selectable heights from a patient's skin is illustrated in FIGS. 19-23. In embodiments, system 55 comprises an anchor 50 rigidly couplable to an anatomical landmark, the anchor comprising a shaft 50a extending along an axis and a reference marker 58 comprising a marker body 10 having an aperture 10e extending through the marker body for slidably receiving the anchor shaft 50a, the marker body comprising a material having any of color, luminance, or reflectance properties to increase visual contrast of the marker body relative to biologic tissue. In embodiments, the marker body 10 is slidably movable and securable along the anchor shaft 50a the anchor is rigidly attached to the anatomical landmark.

In embodiments, the system 55 further comprises a locking mechanism 65 for securing the marker body 10 to the anchor shaft 50a comprises an aperture extending through the marker body. In embodiments, the locking mechanism 65 comprises a screw 65a movable mounted within a shaft 65b, different from aperture or shaft 10e but in fluid communication therewith. In embodiments, the screw 65a frictionally engages the anchor shaft. In embodiments, the system further comprising a key 62 removably engagable with the screw 65a. In embodiments, the key 62 and the screw 65a have complimentary mating engagement features on exterior surfaces thereof, such as complementary threads. Similarly, the top surface of screw 65a and the end 62c of key 62 may have complimentary mating engagement features such as a particular pattern, e.g. Hex head, Philips head, etc. Further, marker 58 may be implemented substantially the same as anchor 8 except that for locking mechanism, as described herein, and aperture 10e of marker 8 are modified in marker 58 to accommodate the circular cross-sectional shape of either shaft 40a or 50a.

In use, a practitioner would rigidly couple an anchor to an anatomical landmark, secure a reference marker to the anchor shaft at or above skin of a patient by slidably receiving the anchor shaft in the maker body and thereafter secure the reference marker to the anchor shaft with a key mechanism by advancing the screw of the locking mechanism through the marker body until contacting the anchor shaft. The process is reversed for removing the system from the patient.

In embodiments the key head 62 be may comprise a material having any of color, luminance, or reflectance properties to increase visual contrast of the marker body relative to biologic tissue and may also have a shape that is not symmetrical in any two or three axis by which the key head is observable.

In some implementations, marker 8 may have an adhesive element disposed on the underside of surface 10A in addition to or in place of the disclosed securing mechanism 10d to secure the marker body 10 to the patient anatomy. In some implementations, such adhesive is covered by a removable tab 10g to keep the adhesive surface sterile until the time of securing to the patient anatomy or surface of interest. The adhesive may be implemented with a biocompatible adhesive or other adherent capable of relatively easy securing or removal from biologic tissue with causing trauma to the site of attachment.

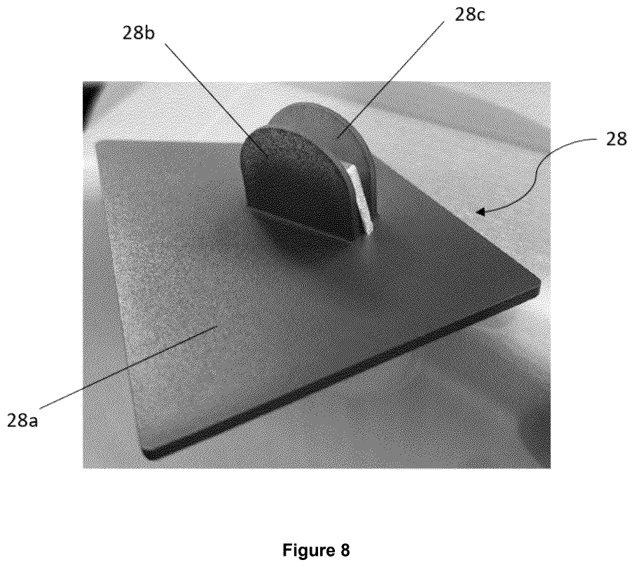

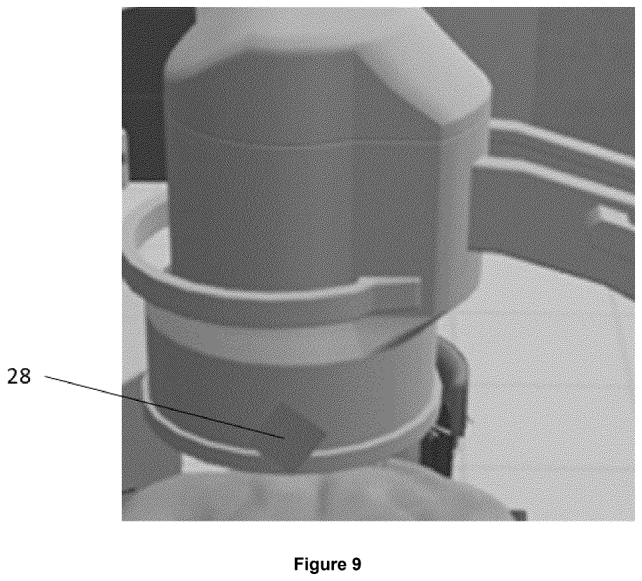

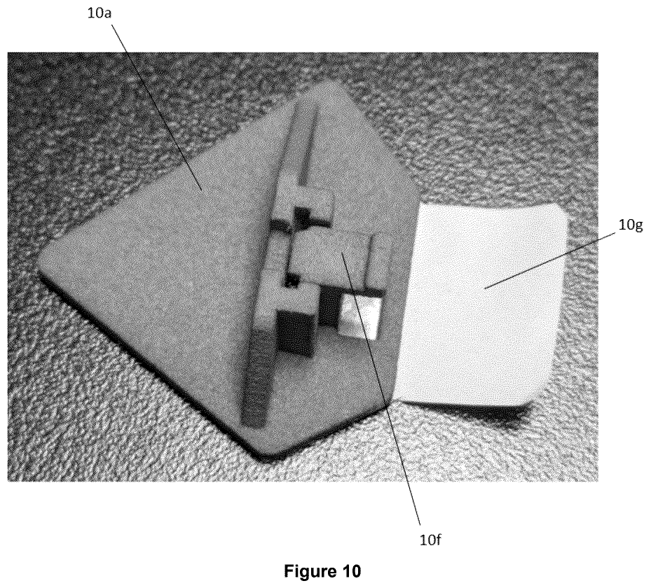

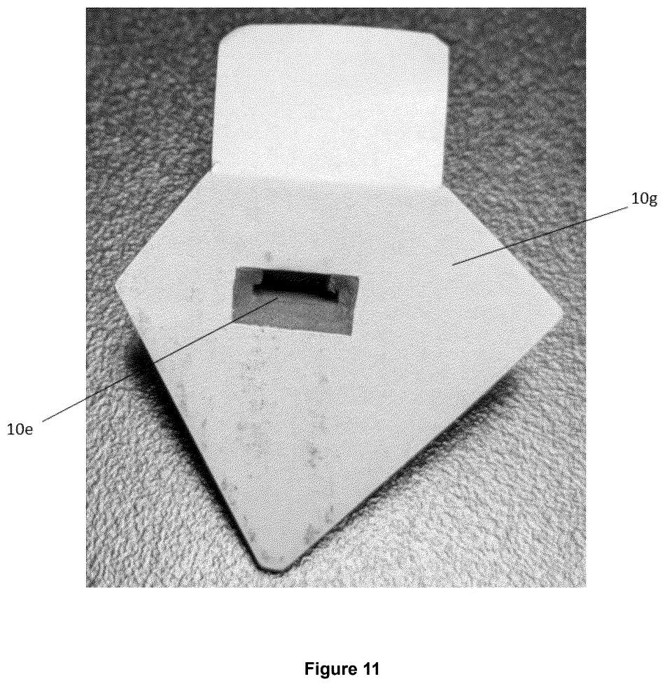

In some implementations, the reference marker may have the configuration of marker 28 of FIG. 8. Marker 28 is substantially similar to reference marker 8 in construction and function, except that projections 28b and 28c, extending outwardly from planar surface 28a, are spaced apart a distance that will allow frictional engagement of the projections 28b and 28c to a surface or lip of an imaging system, such as the radiation detector housing of a C-arm X-ray machine, as illustrated in FIG. 9. In this manner, when viewed by a color or visible light camera, the unique geometry and surface texture of marker 28 enables the marker 28 to be easily distinguished from its surroundings, regardless of a camera angle(s), to enable precise tracking of the position and orientation of marker 28 and the radiation detector in a three-dimensional space.

In some implementations, marker 28 may have a biocompatible adhesive or other adherent disposed on a surface thereof in addition to or in place of projections 28b and 28c to secure the marker body to a surface or lip of a radiation detector.

In some implementations, any of reference markers 8, 28, or 48 may be made at least partially of radiopaque material to be distinctly identifiable in medical imaging data captured by radiographic images.

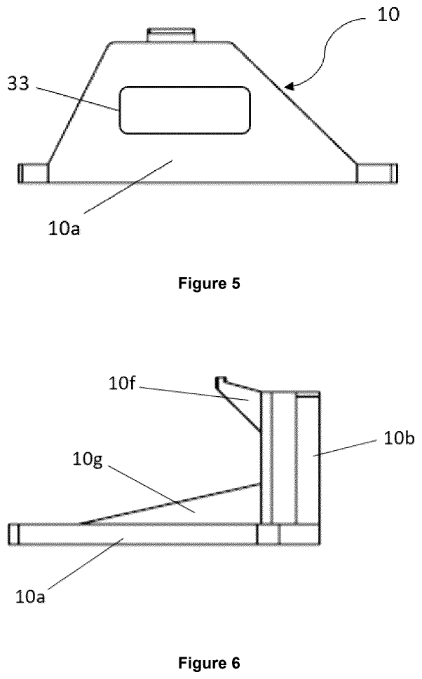

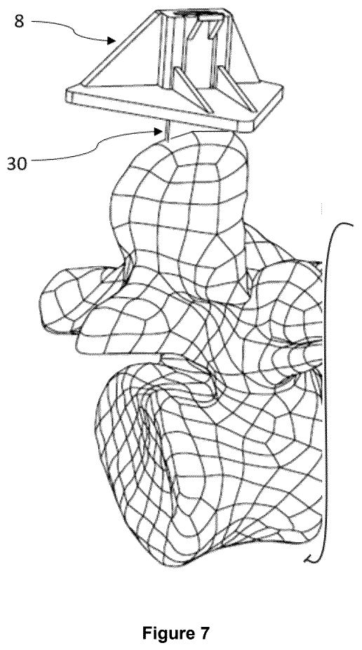

In some implementations, any of reference markers 8, 28, or 48 may be used as a source of information relating to the object of interest. e.g. patient, to which it is attached, or other related information. Specifically, as shown in FIG. 5, reference marker 8 further includes a data structure 33 implemented in the form of a readable label, film or tag, attached to a surface of the reference marker. In some implementations, data structure 33 may be implemented with a QR code which is a machine-readable code that includes an array of black and white regions, typically used for storing URLs or other information for reading by the camera, smartphone or a code scanner. The QR code may be used to convey data about the marker itself, including the model, the patient, the procedure, calibration information relating to the radiation equipment, or other data about the marker, such as a serial number for the marker for establishing authenticity in terms of commercial source, and tracking production details such as the lot or batch number, expiration date, etc. and which may be used in communication with an electronic device to read the results. Other data structure formats such as two and three-dimensional barcodes may be similarly utilized for data structure 33 with the reference markers disclosed herein. In some implementations, of the foregoing information may be simply printed on the label itself for visual examination.

In some implementations, one or more markers 8, 28 or 48 and one or more anchors 30 may be packaged and sold, in any combination thereof, as a kit containing instructions. For example, a pair of markers 8 and 28 may be packaged with one or more anchors 30 as a kit in sterile packaging along with an instructional materials on how to utilize the devices. Alternatively, multiple markers or multiple anchors may be sold as a kit.

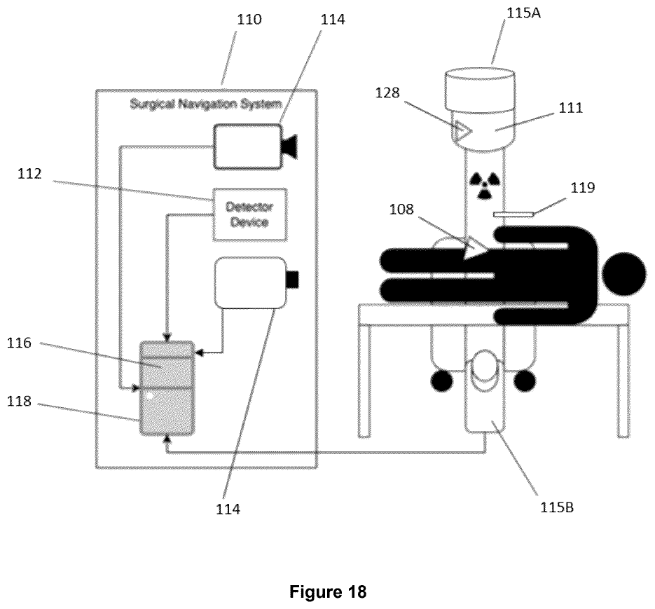

Any of the disclosed markers 8, 28 or 48 may used as part of a medical imaging and surgical navigation system 110, described in FIG. 18. In such systems, the marker 8 provides an exact reference point within three-dimensional space which can be used to enhance registration of virtual and physical matrixes by providing a reliable reference point.

FIG. 18 illustrates conceptually a surgical navigation system 110 suitable for use with the reference markers and anchors described herein. The surgical navigation system 110 may be used with a traditional fluoroscopy machine, e.g. a C-arm, having a source of radiation section and a radiation detector section. The surgical procedure space usually includes a surface on which the patient is disposed. A source of fluoroscopic radiation or x-rays is disposed beneath the surface on one side of the patient, as illustrated, while a detector of fluoroscopic radiation or x-rays is disposed on the opposite side of the patient.

In some implementations, surgical navigation system 110 comprises reference markers 108 or 128, a radiation detector 112, a calibration target 111 attached to an image acquisition device 115A, cameras 114, computer 116, and a display interface 18 and is used with an radiation source 115B and radiation detector/acquisition device 115A, to provide a display of positional images of the patient anatomy and any surgical tools 119 as determined relative to a the reference markers 108 and 128. In some implementations, the components of surgical navigation system 110 may be contained within a single housing which is easily positionable along three axes within the surgical procedure space. Alternatively, one or more the components of surgical navigation system 110 may be located remotely from other components but interoperable therewith through suitable network infrastructure.

The surgical system 110 and particularly cameras 114 track the reference marker 108 or 128 within the camera coordinate system, and forward the positional information of the reference marker onto computer 116 for further processing.

Although the reference markers and anchors disclosed herein have been described with reference to patient anatomy and surgical procedures, their applicability is not limited to the same. Any of the reference markers disclosed herein may be utilized in other situations, including industrial control, package or baggage handling, or any other environments in which says position and tracking of objects is required.

At various places in the present specification, values are disclosed in groups or in ranges. It is specifically intended that the description includes each and every individual sub-combination of the members of such groups and ranges and any combination of the various endpoints of such groups or ranges. For example, an integer in the range of 0 to 40 is specifically intended to individually disclose 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, and 40, and an integer in the range of 1 to 20 is specifically intended to individually disclose 1, 2, 3, 4, 5, 6, 7, 8, 9, 10, 11, 12, 13, 14, 15, 16, 17, 18, 19, and 20.

For purposes of clarity and a concise description, features are described herein as part of the same or separate some implementations, however, it will be appreciated that scope of the concepts may include some implementations having combinations of all or some of the features described herein. Further, terms such as “first,” “second,” “top,” “bottom,” “front,” “rear,” “side,” and other are used for reference purposes only and are not meany to be limiting.

The above detailed description includes references to the accompanying drawings, which form a part of the detailed description. The drawings show, by way of illustration, specific some implementations in which the invention can be practiced. These some implementations are also referred to herein as “examples.” Such examples can include elements in addition to those shown or described. However, the present inventors also contemplate examples in which only those elements shown or described are provided. Moreover, the present inventors also contemplate examples using any combination or permutation of those elements shown or described (or one or more aspects thereof), either with respect to an example (or one or more aspects thereof), or with respect to other examples (or one or more aspects thereof) shown or described herein.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In this document, the terms “including” and “in which” are used as the plain-English equivalents of the respective terms “comprising” and “wherein.” Also, in the following claims, the terms “including” and “comprising” are open-ended, that is, a system, device, article, composition, formulation, or process that includes elements in addition to those listed after such a term in a claim are still deemed to fall within the scope of that claim. Moreover, in the following claims, the terms “first.” “second,” and “third,” etc. are used merely as labels, and are not intended to impose numerical requirements on their objects.

The following claims are hereby incorporated into the Detailed Description as examples or some implementations, with each claim standing on its own as a separate embodiment, and it is contemplated that such some implementations can be combined with each other in various combinations or permutations. The scope of the invention should be determined with reference to the appended claims, along with the full scope of equivalents to which such claims are entitled.

What is claimed is:

1. A system for securing a reference marker to a patient's skin or at user selectable heights from a patient's skin, the system comprising:
an anchor rigidly couplable to an anatomical landmark, the anchor comprising a shaft extending along an axis; and
a reference marker comprising a marker body having an aperture extending therethrough for slidably receiving the anchor shaft, the marker body comprising a material having any of color, luminance, or reflectance properties to increase visual contrast of the marker body relative to biologic tissue;
wherein the marker body is slidably movable and securable along the anchor shaft axis while the anchor is rigidly attached to the anatomical landmark.

2. The system of claim 1 further comprising a locking mechanism for securing the marker body to the anchor shaft.

3. The system of claims 2 wherein the locking mechanism comprises an aperture extending through the marker body.

4. The system of claim 2 wherein the locking mechanism further comprises a screw movable mounted within the marker body.

5. The system of claim 2, wherein the locking mechanism frictionally engages the anchor shaft.

6. The system of claims 2 further comprising a key removably engagable with the locking mechanism.

7. The system of claim 6 wherein the key and the locking mechanism have complimentary mating engagement features.

8. A reference marker securable to a patient's skin or at user selectable heights from a patient's skin, the reference marker comprising:
a marker body comprises material having any of color, luminance, or reflectance properties to increase visual contrast of the marker body relative to biologic tissue,
a first hollow shaft extending through the marker body and configured for slidably receiving a surgical anchor shaft therein; and
a second hollow shaft extending through the marker body and in communication with the first hollow shaft and configured for movably receiving a securing device therein.

9. The reference marker of claim 8 in combination with surgical anchor.

10. The reference marker of claim 9 wherein the surgical anchor is rigidly couplable to an anatomical landmark, the anchor comprising a shaft and a plurality of attachment features disposed along the anchor shaft, wherein each respective attachment feature provides a possible point of attachment of the reference marker to the anchor shaft at a different user selectable heights above skin of the patient.

11. The reference marker of claim 8 wherein the securing device and the second hollow shaft have complimentary mating features on respective exterior surfaces thereof.

12. The reference marker of claim 8 wherein the second shaft has a threaded exterior and the shaft and the securing device comprises a threaded screw movable disposed within the second shaft.

13. The reference marker of claim 8 further in combination with a key receivable within an end of the screw for advancing or retracting the screw within the second shaft.

14. The reference marker of claim 8 wherein the anchor shaft and first hollow shaft have a substantially circular cross-sectional shape.

15. A method for identifying a point of interest in medical imaging procedures, comprising:
A) rigidly coupling an anchor to an anatomical landmark, the anchor comprising a shaft extending along an axis;
B) securing a reference marker to the anchor shaft at or above skin of a patient, a reference marker comprising a marker body having an aperture extending therethrough for slidably receiving the anchor shaft, the marker body comprising a material having any of color, luminance, or reflectance properties to increase visual contrast of the marker body relative to biologic tissue; and
C) detecting a position of the reference marker.

16. The method of claim 15, wherein A) comprises:
A1) securing the anchor in a position of a patient's anatomy proximate a surgical entry point.

17. The method of claim 15, wherein the marker body further comprises a securing mechanism movably mounted therein and A) further comprises:
B1) securing the reference marker to the anchor shaft with a key mechanism by advancing the securing mechanism through the marker body until contacting the anchor shaft.

18. A system for identifying a point of interest in a medical procedure, the system comprising:
an anchor rigidly couplable to an anatomical landmark, the anchor comprising a shaft extending along an axis and further a plurality of attachment features disposed along the anchor shaft, wherein each respective attachment feature provides a possible point of attachment of the reference marker to the anchor shaft at a different user selectable heights above skin of the patient;
reference marker comprising a marker body having an aperture extending therethrough for slidably receiving the anchor shaft;
securing mechanism movably disposed within the marker body for attaching the marker body at a different positions along the axis of the anchor shaft.

19. The system of claim 18 wherein securing mechanism frictionally engages the anchor shaft.

20. The system of claim 18 wherein securing mechanism comprises a screw disposed within a threaded shaft in the marker body, the threaded shaft configured to movably receive the screw therein.

21. The system of claim 18 in combination with a key mechanism for releasably engaging and disengaging the screw to or from the anchor shaft.

22. The system of claim 18 wherein the key mechanism and screw have complimentary mating features on respective exterior surfaces thereof.
